(19) United States
(12) Patent Application Publication
(10) Pub. No.: US 2007/0053951 A1
(43) Pub. Date: Mar. 8, 2007

(54) COMPOSITE BIOMATERIAL FOR BONE IMPLANTS

(75) Inventors: Ramon Gonzalez Santos, Ciudad Habana (CU); Alberto Guillermo Suzarte Paz, Ciudad Habana (CU)

Correspondence Address: AXIOS LAW GROUP, 1725 WESTLAKE AVENUE NORTH, SUITE 150, SEATTLE, WA, 98109, US

(73) Assignee: Centro Nacional de Investigaciones Cientificas, Ciudad de la Habana (CU)

(21) Appl. No.: 10/595,411

(22) PCT Filed: Sep. 30, 2004
(86) PCT No.: PCT/CU04/00009

§ 371 Date: Sep. 12, 2006

(30) Foreign Application Priority Data
Oct. 16, 2003 (CU) CU 2003-0237

(51) Int. Cl. A61F 2/02

(52) U.S. Cl. 424/423

(57) ABSTRACT

Biomaterials for bone implants constituted by an inorganic phase formed by calcium salts, mainly phosphates, hydroxy- and carbonate-apatite and an organic phase formed by polymers obtained from vinyl acetate and crotonic acid. These biomaterials can be dense or porous and present different speeds of resorption when they are used as implants in the live tissue depending on the nature and proportion of the phases and they can also work as supports for the production of controlled drug delivery systems.

### FIELD

This invention is applied in the field of synthesis and application of biomaterials, in particular those well-known as composed or mixed biomaterials (“composites”) in this case formed by ceramic of calcium carbonate-phosphates and derivative polymers of the vinyl acetate able to act as substitutes of the hard tissue when it has been damaged or lost or other biomedical applications as drug supports, controlled drug delivery systems, etc.

### BACKGROUND

The need to substitute, reconstruct and/or regenerate the damaged or lost bone tissue in different places of the human body has been a challenge faced with more or less success along the centuries since the very beginning of humanity. Although one of the first solutions found was the use of different types of bone graft, nowadays they are being substituted by other biomaterials due to their diverse limitations, related mainly to the difficulty in their obtainment, trouble and surgical risks for the patients and variable effectiveness among others. The materials employed for these purposes make a long list that includes different types of metals, polymers, ceramic and glasses that are known generically as bone graft substitutes (Senn N. On the healing of aseptic cavities by implantation of antiseptic decalcified bone. Amer. J. Med. Sc., 98: 219-243; 1889., Weber J. M., White E. W. it Carbonates minerals ace precursors of new ceramic, metal and polymer materials for biomedical applications. Min. Sci. Eng., 5: 151; 1973, Williams D. F. Challenges in materials for health care applications. Argew. Chem. Adv. Mater., 101(5): 678; 1989, Hench L. L., Wilson T. Surface-activate biomaterials. Science, 226: 630; 1984). It can be said that until now the ideal biomaterial that suits all the exigency of the bone reconstructive surgery for different medical specialties has not been found, however, there is a widespread consent among the specialists, that from the point of view of their biocompatibility, tolerance by the human body and effectiveness in the cure, the calcium phosphates and in particular the hydroxyapatites are the most promising biomaterials in this field (Klein C. P. A. T., Dreissen A. A., of Groot K., Van den Hooff A., Biodegradation behavior of various calcium phosphate materials in bone tissue. J. Biomed. Mater. Head., 17: 769-784; 1983, Shinazaki K., Mooney V., Comparative study of porous hydroxyapatite and calcium material phosphate in bone substitute. J. Orthop. Head., 3: 301; 1985, Damien C. J., Parsons J. R. Bone graft and bone graft substitutes: TO review of current technology and applications. J. Appl. Biomat., 2:187-208; 1991, Jallot E. Correlation between hydroxyapatite osseointegration and Young's Modulus. Med. Eng. Phys., 20: 697; 1998). It is it because these substances present a great chemical and structural identity with the mineral support of the bone, well known as the “biological apatite.” That is the reason why these compounds have become successful in the clinical use, mainly in the last 30 years (Cottrell D. A., Wolford L. M. Long-term evaluation of the uses of coralline hydroxyapatite in orthognatic surgery. J. Oral Maxillofac. Surg., 56: 935; 199,. Ayers R. A., Simske S. J., Nunes C. R., Wolford L. M. Long-term bone ingrowth and residual mocrohardeness of porous block hydroxyapatite implants in human. J. Oral Maxillofac. Surg., 56: 1297; 1998, Gonzalez R., Blardoni F., Master H., Pereda O., Pancorbo E., Cienega M. A. Long-term results of the coralline porous hydroxyapatite HAP-200 ace bone implant's biomaterial in orthopedics and traumatology. CENIC Biological Sciences have, 32(2): 97-101; 2001).

But the research carried out by different specialists have allowed to check that the bone is formed by an inorganic support (approximately 65%) constituted mainly by these calcium phosphates above mentioned and the rest (35%) is organic matter and water. The organic phase is for the most part made up of collagen found in a close interrelation with the biological apatite (Weiner S., Traub W. Organization of hydroxyapatite crystals within collagen fibrils. FEBS, 206(2): 262; 1986).

Knowledge of the composition and structure of the bone tissue has stimulated the research and development of biomaterials of calcium phosphates with different composition, structure, porosities and with different biodegradation behaviors “in vivo”. Nowadays there is a great variety of this type of implants among them those described in the patents U.S. Pat. No. 4,976,736 (Dec., 1990), U.S. Pat. No. 5,900,254 (May, 1999), FR 2776282 (Sept. 1999), U.S. Pat. No. 6,001,394 (Dec. 1999), U.S. Pat. No. 6,331,312 (Dec. 2001). Natural polymeric biomaterials (collagen, quitosane, cellulose, etc) as well as synthetic from different nature and origin, simulating the organic part of the bone have also been developed and applied with these purpose [U.S. Pat. No. 5,837,752 (Nov. 1998), U.S. Pat. No. 5,919,234 (Jul. 1999), U.S. Patent Application 2003114552 (Jun. 2003)]. More recently an intense work on the production of composed or mixed materials (composite), made up by calcium phosphates and hydroxyapatite with different types of natural and synthetic polymers is being done with the purpose of achieving products with chemical, physical and mechanical properties more similar to the bone and with which a better functional acting as substitutes of the bone graft can be achieved.

A great number of combinations of this type of composed materials (composite) has been developed in the last years, among them can be mentioned the following: hydroxyapatite, collagen and a glycosamineglicane (U.S. Pat. No. 5,071,436, 1991), mixture of calcium phosphates with cellulose (FR2715853, 1995), hydroxyapatite with polilactic acid (WO9746178, 1997), hydroxyapatite with silicone (U.S. Pat. No. 5,728,157, 1998), calcium phosphate with cellulose and its derivates (U.S. Pat. No. 6,558,709, 2003), several salts of calcium with several formulations of polymers (U.S. Pat. No. 6,579,532, 2003), hydroxyapatite, bone and several inorganic salts with polyethyleneglycol, waxes, hydrogels and acrylic latex (U.S. Pat. No. 6,605,293, 2003).

No report on possible combinations of derivative polymers of vinyl acetate and crotonic acid with any of the inorganic salts possible to be used as implant biomaterial was found.

Nevertheless, although the above products have shown good results in some medical applications, they don't still satisfy the diverse and growing need of the reconstructive surgery of the bones of different regions of the human body.

For some applications the surgeons prefer the biomaterial to be implanted reabsorb quickly leaving new bone in its place, in other cases it is demanded that the implant remains unchangeable for longer period of time according to the place and magnitude of the lesion to treat. Additionally it is very favorable if the biomaterial is able to deliver drugs in a controlled way, because it allows to treat different pathologies of the bone like infections, inflammatory processes, neoplacy, etc. at the same time.

On the other hand, many of the existing biomaterials for bone implants are not economically viable to apply them in mass population.

Recently derivative polymers from vinyl acetate and crotonic acid able to act as appropriate supports for the production of medicament of sustained action have been developed (Cuban Patents No. 22199 of 1993, 22880 of 2003, Int. Appl. PCT/CU99/00002 1999).

### DETAILED DESCRIPTION

The present invention also involves new applications of these polymers in the production of implantable biomaterials in humans to reconstruct the bone tissue and for other therapeutic applications or reconstructive surgery.

The main objective of this invention is to obtain composed or mixed biomateriales (“composite”) made up of calcium phosphates, carbonates and hydroxide, hydroxyapatite, carbonate-apatite or their mixtures in different proportions joined to derivative polymers from vinyl acetate and crotonic acid with appropriate properties to work as substitutes of bone implant.

Another objective of this invention is that the developed biomaterials can be dense or porous and with different biodegradation grades according to surgical necessities for the place, type of bone and magnitude of the lesion to treat.

We also search for that these biomaterials be formed by successive layers ceramic-polymer-ceramic or polymer-ceramic-polymer in such a way that surfaces of contact of the biomaterial with the live tissue can be obtained only formed by the polymer, by the ceramic or by both.

As application examples, clarifying the carrying out of our invention we give the following:

The developed biomaterials are made up of two types of compounds; an inorganic phase (A) and an organic phase (B).

The inorganic phase (A) it is constituted by calcium phosphates, hydroxide, carbonates, hydroxyapatite and carbonate-apatite or a mixture of them in different proportions as it is shown in the following chart:

CHART 1
Composition of some mixtures of inorganic compounds
(A) employed in the preparation of biomaterials.

| | Caa(PO4)b(H)c(CO3)d(OH)e | | | | | |
|---|---|---|---|---|---|---|
| Mixtures | (approximated value of the subindexes) | | | | | Molar ratio |
| No. | a | b | c | d | e | Ca/P |
| 1 | 3 | 2 | 0 | 0 | 0 | 1.5 |
| 2 | 10 | 6 | 0 | 0 | 2 | 1.66 |
| 3 | 10 | 6 | 0 | 1 | 0 | 1.66 |
| 4 | 96 | 59 | 4 | 9 | 1 | 1.62 |
| 5 | 100 | 3 | 0 | 95 | 1 | 33 |
| 6 | 44 | 33 | 16 | 2 | 1 | 1.3 |
| 7 | 9 | 5 | 1 | 1 | 2 | 1.8 |
| 8 | 9 | 4 | 2 | 2 | 2 | 2.25 |
| 9 | 10 | 3 | 0 | 5 | 1 | 3.3 |
| 10 | 9 | 6 | 2 | 0 | 2 | 1.5 |

The organic phase (B) is constituted by breakups of poly-vynilacetate-co-vynil alcohol (POVIAC) of composition between 1 and 25% molar of monomeric units of vynil alcohol, of molecular mass and purity similar to the one described for the polyvynilacetate; poly-vinylacetate of molecular mass between 10 000 and 250 000 D, with content of monomer residual between 0 and 100 ppm, acidity up to 0.5% referred to acetic acid, content of heavy metals referred to lead lower that 20 ppm and free from peroxides (POVIAC1); polyvynilacetate co crotonic acid with composition of crotonic acid between 1 and 40% in weight of monomeric units of crotonic acid, monomer content between 0 and 100 ppm, molecular mass between 10 000 and 25 000 D and free from peroxides (CROTAV) or mixture of them in ethanol or acetone in variable concentrations according to the percentage of the polymer wanted to be incorporated to the biomaterial.

### EXAMPLE 1

A homogeneous mixture of calcium salts with an approximate composition to the represented in No. 10 (chart 1) with a mean size of particles of 0.1 mm, was moistened gradually with a solution of POVIAC (25%) in acetone until obtaining a paste. The mixture was sieved to obtain particles between 1 and 2 mm of diameters and was left to dry at room temperature. The dried granule was put to grind in a commiseration mill and the obtained powder was moistened again with acetone, sieved and dried to obtain a compact granulated with a mean size of the particles from 1 to 2 mm.

The product thus obtained with a content of approximately 20% of the polymer is appropriate as material of bone implant for the filling of cavities in the bone such as the sequels of tumors and cysts.

### EXAMPLE 2

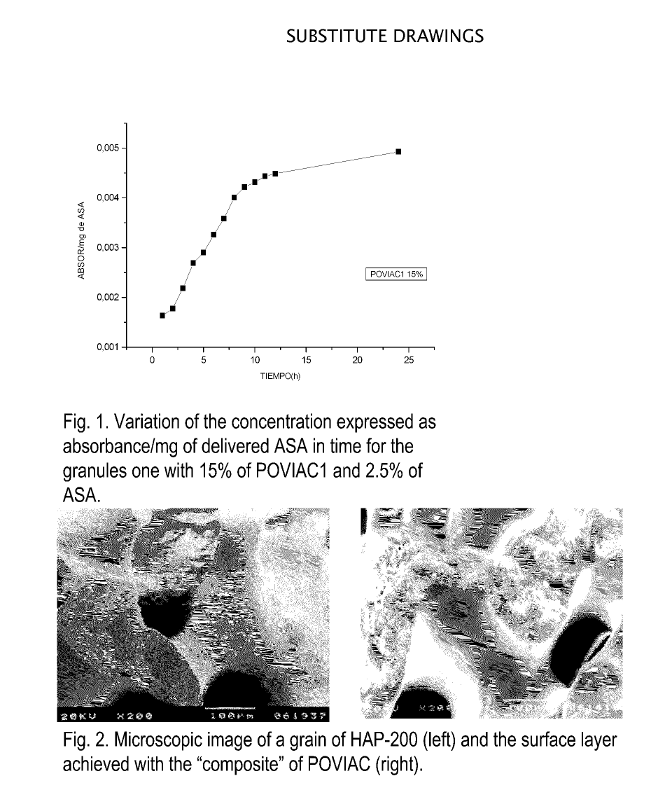

To the mixture of inorganic salts described in the previous example was added acetyl salicylic Acid (ASA) and then mixed appropriately to obtain a homogeneous mass, it was proceeded in the same way to obtain a granule with 15% of POVIAC 1 and 2.5% of ASA. This “composite” behaves as a system for controlled delivery of the drug (FIG. 1).

### EXAMPLE 3

A portion of granules of porous hydroxyapatite HAP-200 (Gonzalez R.; Melo M. C.; Pérez A.; Rodriguez B. C. Coralline Porous Hydroxyapatite HAP-200. Main Physical-chemical characteristic. Quimica Nova, 16 (6) November-December: 509-512; 1993) with particle size between 2 and 2.5 mm and with similar composition to the mixture 2 is submerged in a suspension in acetone of 25% of POVIAC and 10% of mixture No. 5 (chart 1) with size of particles of 0.1 mm. is stirred during 10 min. The granule is separated and dried in the air. The product obtained in this way keeps its original porous structure with a polymer layer stuck in the whole surface (FIG. 2).

### EXAMPLE 4

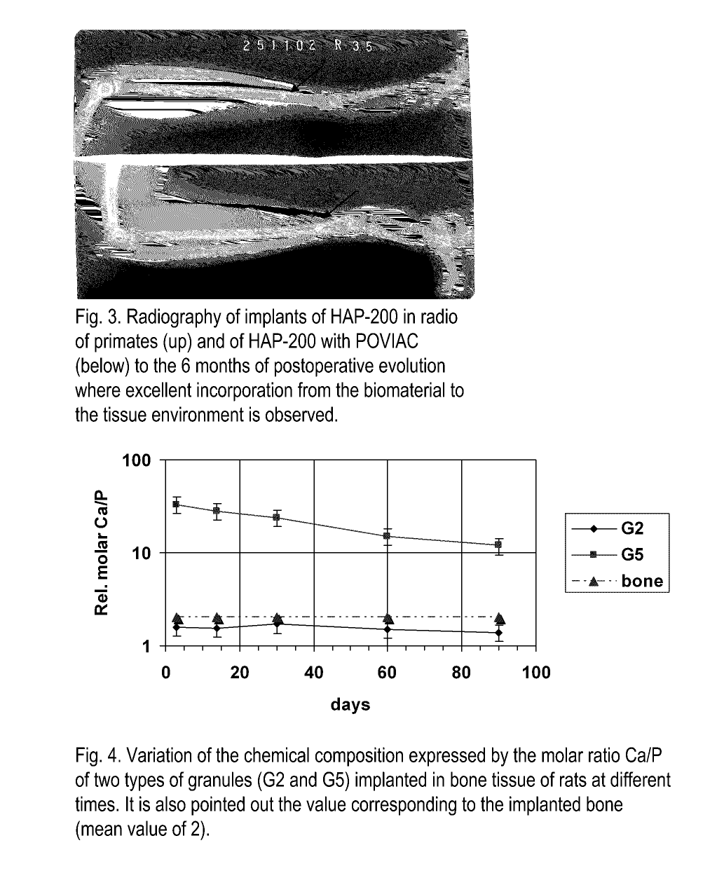

The product obtained in the previous example was tested by means of implants in bone in the superior extremities of primates after being sterilized with gamma radiation at 25 Kgy, showing excellent results in the bone repair without adverse local or general response after 18 months (FIG. 3).

### EXAMPLE 5

A homogeneous mixture of calcium salts with an approximate composition to the represented in No. 10 (chart 1) with a mean size of the particles of 0.1 mm, was moistened gradually with a solution of CROTAV (26%) in ethanol until obtaining a paste. The mixture was sieved to obtain particles between 1 and 2 mm of diameters and left to dry at room temperature. The dried granule was milled in a commiseration mill and the obtained powder was moistened again with ethanol, sieved again and dried to obtain a compact granule with mean size of the particles from 1 to 2 mm.

The product thus obtained with a content of approximately 15% of the polymer is appropriate as material for bone implant for the filling of cavities in the bone such as the sequels of tumors and cysts.

### EXAMPLE 6

Two types of granule prepared according to the procedure described in example 5, one with a similar composition to that of the mixture 2 (G2) and another with that of the mixture 5 (G5) were implanted in bone tissue of rats (femur). The first on the left side and another on the right side after being sterilized with gamma rays at 25 Kgy. The implants were removed and analyzed at different times of postoperative evolution, being determined the variation in the composition of phases by means of FTIR spectroscopy and variation of molar ratio Ca/P by chemical analysis. It was found that while granule G2 did not change its composition considerably in 90 days, granule G5 degradated relatively quickly incorporating phosphorous to its structure to come closer to the half composition of the bone (FIG. 4).

The present invention presents the following advantages:
(1) Physically and mechanically very stable biomaterials are obtained without the crumbling or loosening of isolated particles.
(2) The obtained biomaterials can be dense or porous and they present appropriate biomechanical properties to work as bone graft substitutes.
(3) Biomaterials with different degradation speeds in correspondence with the composition of the present phases are obtained.
(4) The developed biomaterials also work as controlled drug delivery systems, with which is possible to restore the damaged or lost bone at the same time and to treat different bone pathologies with drugs like antibiotics, antinflammatories, etc.
(5) The obtained biomaterials have such porosity that they form a viable matrix for the growth of new tissue in their interior when they are implanted either in soft tissue or in the bone without forming “sack bottom.”

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. Composite biomaterials for bone implants, characterized for having a multiphase chemical composition including an inorganic phase made of calcium salts and another organic phase constituted by polymers or copolymers or both derivates of the vinyl acetate and the crotonic acid.

15. Composite biomaterials for bone implants, according to claim No. 14, characterized for presenting dense or porous structures indistinctly, the latter with pores three-dimensionally interconnected with diameters from 5 to 840 μm.

16. Composite biomaterials for bone implants, according to claim No. 15 characterized because the inorganic phase is constituted by calcium phosphates, hydroxide, carbonates, hydroxyapatite and carbonate-apatite or their mixtures in different proportions in which the molar ratio P/Ca varies between 0 and 99.9.

17. Composite biomaterials for bone implants, according to claim No. 16, characterized because the inorganic salts can be of natural or synthetic origin, dense or porous and physically conformed in order to obtain the desired physical form either in blocks or granules.

18. Composite biomaterials for bone implants, according to claim No. 15 characterized for having in their composition a proportion of the organic phase between 0.1 and 99%, which is constituted by poly-vinyl acetate-co-vinyl alcohol composition between 1 and 25% molar of monomeric units of vinyl alcohol of molecular mass and purity similar to the one described for the polyvinyl acetate.

19. Composite biomaterials for bone implants, according to claim No. 15 characterized for having in their composition a proportion of the organic phase between 0.1 and 99%, which is constituted by poly-vinyl acetate of molecular mass between 10 000 and 250 000 D, with content of residual monomer between 0 and 100 ppm, inferior acidity to 0.5% referred to acetic acid, content of heavy metals referred to lead smaller that 20 ppm and free from peroxides.

20. Composite biomaterials for bone implants, according to claim No. 15 characterized for having in their composition a proportion of the organic phase between 0.1 and 99%, which is constituted by composition of crotonic acid between 1 and 40% in weight of monomeric units of crotonic acid, monomer content between 0 and 100 ppm, molecular mass between 10 000 and 25 000 D and free from peroxides or mixtures of them.

21. Composite biomaterials for bone implants, according to claim No. 17 characterized for having in their composition a proportion of the organic phase between 0.1 and 99%, which is constituted by mixtures of derivative polymers of the vinyl acetate and crotonic acid.

22. Composite biomaterials for bone implants, according to claim No. 18 characterized for a composition which is constituted by mixtures of derivative polymers of the vinyl acetate and crotonic acid.

23. Composite biomaterials for bone implants, according to claims No. 17 or 19 characterized because the organic phase can be homogeneous and evenly distributed in the whole volume of the solid.

24. Composite biomaterials for bone implants, according to claim No. 18 characterized because the organic phase can be homogeneous and evenly distributed in the whole volume of the solid.

25. Composite biomaterials for bone implants, according to claims No. 17 or 19 characterized because the organic phase can be homogeneous and evenly distributed all over the body of the inorganic support.

26. Composite biomaterials for bone implants, according to claim No. 18 characterized because the organic phase can be homogeneous and evenly distributed all over the body of the inorganic support.

27. Composite biomaterials for bone implants, according to claims No. 21 or 22, characterized because when the organic phase is covering the surface of the inorganic support, such surface is formed by successive layers ceramic-polymer-ceramic or polymer-ceramic-polymer in such a way that surfaces of contact of the biomaterial with the live tissue can be formed only by the polymer, by the ceramic or by both.

28. Composite biomaterials for bone implants, according to claim 14, characterized for presenting different speeds of reabsorption between 10⁻⁵ and 3.2% per day, when it work as bone implant depending of the chemical nature and the relationship among the inorganic phases present in their composition.

29. Composite biomaterials for bone implants, according to claims 14, characterized by their use as controlled drug delivery system when they are loaded with the corresponding drug and are implanted in soft tissue as well as in bone.
